(19) United States
(12) Patent Application Publication
(10) Pub. No.: US 2021/0179511 A1
(43) Pub. Date: Jun. 17, 2021

(54) Process To Produce Distillate From Light Alkanes

(71) Applicant: ExxonMobil Research and Engineering Company, Annandale, NJ (US)

(72) Inventors: Kun Wang, Branchburg, NJ (US); Jihad M. Dakka, Whitehouse Station, NJ (US)

(21) Appl. No.: 17/077,059

(22) Filed: Oct. 22, 2020

Related U.S. Application Data
(60) Provisional application No. 62/948,878, filed on Dec. 17, 2019.

(51) Int. Cl. C07C 1/22; C07C 45/27; C07C 45/68; C07C 29/38; B01J 19/24

(52) U.S. Cl. CPC C07C 1/22; C07C 45/27; C07C 45/68; C07C 2529/70; B01J 19/245; B01J 2219/00038; C07C 29/38

(57) ABSTRACT

A method may include reacting at least a naphtha range alkane with oxygen and to produce oxygenate products; reacting at least a portion of the oxygenate products to produce condensed products; and reacting at least a portion of the condensed products with at least hydrogen to produce a distillate range product.

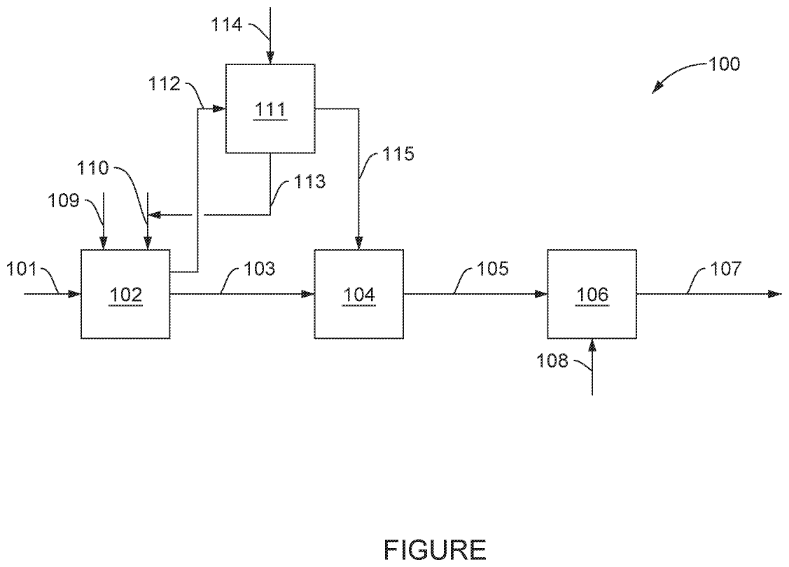

### FIELD

This application relates to a process to convert light alkanes to distillate range products via oxygenate intermediates.

### BACKGROUND

Production of unconventional oil and gas reserves such as tight oil and shale have increased the availability of light alkanes in most markets. As supplies of LPG (liquefied petroleum gas) and light naphtha have become more abundant, the cost of these lighter hydrocarbons has generally declined. These lighter hydrocarbons are typically not well suited for use as fuels but are rather used as lower cost feed stocks to produce petroleum-derived chemicals.

While chemical production is expected to shift to the lower-cost, lighter hydrocarbon feedstocks, increased chemical production from lighter alkanes may not address the surplus of lighter alkanes in the market currently and from the expected increase in supply as more unconventional wells are drilled and come into production. Further, while demand for motor gasoline is expected to increase at reduced rates or even flatten and decrease in the long term, demand for distillate range products such as jet fuel and diesel fuel is expected to continue to grow. Additionally, as automotive manufacturers switch to high-performance turbo-charged engines to meet efficiency and emissions regulations, demand for higher octane fuels will increase which will limit the blend volume in motor gasoline for lower octane molecules such as n-alkanes. For these and other market forces, there is a desire in the industry to convert lighter alkanes to distillate range products.

There have been previous efforts to convert light alkanes to distillate range products, but most processes require olefin intermediates. A typical process may include stream cracking of light alkanes such as LPG or naphtha to produce C₂-C₅ olefins, or catalytic dehydrogenation of C₃-C₉ alkanes to produce C₃-C₉ olefins, followed by oligomerization of olefins to produce distillate range products. However, the olefin generation is endothermic and requires high temperature and low pressure to overcome thermodynamic limitations inherent to the dehydrogenation and cracking processes. The large excess energy required to drive the reactions forward typically makes the resultant distillate range products produced from olefin intermediates economically uncompetitive as compared to producing distillate range products directly from crude oil.

### SUMMARY

Disclosed herein is an example method including: reacting at least a naphtha range alkane with oxygen and to produce oxygenate products; reacting at least a portion of the oxygenate products to produce condensed products; and reacting at least a portion of the condensed products with at least hydrogen to produce a distillate range product.

Further disclosed herein is an example including: introducing a naphtha range alkane feed into an oxidation unit wherein the naphtha range alkane feed comprises naphtha with a boiling point of about 30° C. to about 200° C. at atmospheric pressure; reacting at least the naphtha with oxygen to produce an oxygenate product comprising at least one of an alcohol, a ketone, or combinations thereof; introducing the oxygenate product into a condensation unit; reacting at least a portion of the oxygenate product to produce a condensed product comprising at least one of an olefin, an alcohol, a conjugated enone, or a combination thereof; introducing the condensed products into a hydro-finishing unit; and reacting at least a portion of at least the condensed products with hydrogen to produce a distillate range product.

Further disclosed herein is an example system including an oxidation unit, wherein a naphtha range alkane feed is coupled to one or more inputs of the oxidation unit, and wherein the oxidation unit is configured to oxidize at least a portion of the naphtha range alkane feed to produce an oxidized effluent stream comprising oxygenates; a condensation unit, wherein the oxidized effluent stream from the oxidation unit is coupled to one or more inputs of the condensation unit, and wherein the condensation unit is configured to react at least a portion of the oxygenates from the oxidized effluent stream to produce a condensed product stream comprising condensed products; and a hydro-finishing unit, wherein the condensed product stream effluent from the condensation unit and a hydrogen stream are coupled to one or more inputs of the hydro-finishing unit, and wherein the hydro-finishing unit is configured to react at least a portion of the condensed products with hydrogen to produce a distillate range product stream.

### BRIEF DESCRIPTION OF THE DRAWINGS

These drawings illustrate certain aspects of the present disclosure and should not be used to limit or define the invention.

FIGURE is a flow chart illustrating an embodiment of a process to convert light alkanes to distillate range products.

### DETAILED DESCRIPTION

This application relates to production of distillate range products from light alkanes, and, more particularly, to embodiments related to a methods and systems to produce distillate range products from oxygenate intermediates. While the methods and systems disclosed herein may be suitable to provide distillate range products in a standalone unit, the methods and systems may be particularly suitable for an integrated process within a petroleum refinery or chemical processing plant.

There may be several potential advantages to the methods and systems disclosed herein, only some of which may be alluded to in the present disclosure. One of the many potential advantages of the methods and systems is that the inefficiencies from utilizing on-purpose olefin production for production of distillate range products may be addressed. As discussed above, steam cracking and dehydrogenation may be two processes which produce on-purpose olefins. Catalytic alkane dehydrogenation to produce olefins typically requires high temperatures, low pressure, and frequent catalyst regeneration. Dehydrogenation methods may be limited by low equilibrium conversions due to the endothermic nature of the dehydrogenation reactions. The relatively low-per pass conversion in dehydrogenation methods may lead to a large recycle ratio. Steam cracking of naphtha to produce olefins also requires high temperatures, low naphtha partial pressure, and the reactor may be readily fouled by coking reactions. In either process, process conditions which favor olefin production are high temperature (e.g., >840° F. or 450° C.) and low pressure (ambient or vacuum). These process conditions are often satisfied by supplying large amounts of heat to the reactor to overcome the equilibrium constraint to reach appreciable per-pass olefin conversion. Products of dehydrogenation and steam cracking often require cryogenic separation and compression which adds to the energy requirement of the naphtha to olefins conversion process. In a typical steam cracker, olefins production accounts for approximately one-third of the overall unit operational cost, and olefins separation accounts for approximately two-thirds of the overall unit operational cost. After the olefins are produced either by steam cracking or dehydrogenation, the olefins may be oligomerized to distillate range products. The carbon number distribution of the products may depend on feed composition, catalyst, and process conditions. The distillate range products produced from olefin intermediates may be expensive due to the large energy requirement of olefin production and separation.

Embodiments may include an integrated process for production of distillate range products from naphtha range alkanes via oxygenate intermediates. Naphtha range alkanes suitable for use in the embodiments disclosed herein may include alkanes with carbon numbers from C₅ to C₁₂. Naphtha range alkanes may be acyclic branched or unbranched hydrocarbons having the general formula CₙH₂ₙ₊₂ and cyclic alkanes having the molecular formula of CₙH₂. In some embodiments, naphtha range alkanes may further comprise aromatics, olefins, or dienes in the boiling point range of naphtha cut. Naphtha may be a general term utilized in industry as identifying a cut of hydrocarbon from distillation. Naphtha is typically sourced in refineries from the distillation of crude oil in atmospheric distillation columns. The composition of refinery naphtha may be determined by the location of the distillation cut within the atmospheric distillation column. Some refineries may produce two or more grades of naphtha, sometimes referred to as light naphtha and heavy naphtha. Light naphtha may also be referred to as light straight run naphtha, natural gasoline, light paraffinic naphtha, or pentanes plus, for example. Light naphtha may include relatively lighter hydrocarbons with carbon numbers between C₅ and C₆ such as n-pentane and n-hexane and isomers thereof, typically with boiling points between about 30° C. and about 90° C. at 101.325 kPa. Light naphtha may be sourced from crude oil or from separation of natural gas liquids or from other chemical processes within a refinery. Heavy naphtha may also be referred to as heavy straight run naphtha or reformable naphtha. Heavy naphtha may include relatively heavier hydrocarbons with carbon numbers between C₆ and C₁₂ such as n-hexane, n-heptane, n-octane, n-nonane, n-decane, n-undecane, n-dodecane, and isomers thereof, for example, typically with boiling points between 90° C. and 200° C. at 101.325 kPa. Additionally, heavy naphtha may include cyclic alkanes such as cyclopentane, cyclohexane, which may be unsubstituted or substituted one or more alkyl groups such as methyl, ethyl, n- or iso-propyl, n-, sec- or t-butyl group. Further, heavy naphtha may include aromatic compounds such as benzene, toluene, xylenes, ethylbenzene, cumene, methylethyl benzene, or butylbenzenes, for example. In refineries, heavy naphtha is typically sourced directly from atmospheric distillation of crude oil, however, additional volumes of heavy naphtha may be sourced from hydrocrackers or from cokers as coker naphtha.

Distillate range products may be an industry term used to identify a cut of hydrocarbon products produced in a refinery. Distillate range products may be produced in various units in a refinery such as atmospheric distillation units, vacuum distillation units, alkylation units, catalytic cracking units, hydrodesulfurization units, and hydrotreating units, for example. Distillate range products may have many synonyms including middle distillates or gasoil and may include hydrocarbons which boil in a range of about 180° C. to about 360° C. at 101.325 kPa. Distillate range products may include specific products such as extra light heating oil, distillate fuel oil, diesel fuel, marine diesel oil, jet fuel, and kerosene, for example. The distillate range products produced by the methods disclosed herein may have carbon numbers that are double or triple the carbon numbers of the naphtha range alkanes which the distillate range products are derived from. For example, the distillate range products may have carbon numbers ranging from C₁₂ to C₃₆ such as n-dodecane through n-hexatriacontane and isomers thereof.

The methods and systems described herein may utilize oxygenate intermediates to reduce process severity and energy requirements for producing distillate range products from light alkanes, particularly naphtha range alkanes having carbon numbers from C₅ to C₁₂. The process may include the following steps: (1) oxidation of naphtha to produce oxygenate species; (2) condensation of the oxygenate species to produce condensed species; and (3) hydro-finishing of the condensed species to produce distillate range products. In some embodiments, naphtha range alkanes may be fed to an oxidation unit which may selectively oxidize the naphtha range alkanes to an oxygenate product stream comprising alcohols and ketones with substantially the same carbon number as the naphtha range alkanes. Thereafter, the oxygenate product stream may be fed to a condensation unit which may condense alcohols and ketones from the oxygenate product stream to produce a condensed product stream comprising products with double or triple the carbon numbers of the naphtha range alkanes. Finally, the condensed product stream may be hydro-finished to produce a distillate range product stream.

In Step (1), any suitable technique for oxidation of naphtha to produce oxygenate species may be used. By way of example, the oxidation may include reaction of naphtha range alkanes with oxygen to produce a mixture of ketones and alcohols with carbon numbers corresponding to the carbon numbers of the naphtha range alkanes. Reaction 1 shows a general reaction of a naphtha range alkane and oxygen to produce a ketone and an alcohol and with carbon numbers corresponding to the carbon number of the naphtha range alkane.

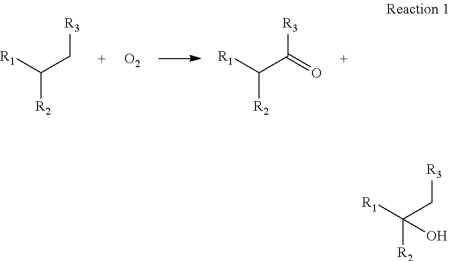

In Reaction (1), corresponding to Step (1) above, the naphtha range alkane may include R₁, R₂, and R₃ substitution groups. R₁ and R₂ may be individually selected from H or a hydrocarbyl group containing 1 to 6 carbon atoms, wherein the hydrocarbyl group is linear or branched. R₃ may be selected from H or a hydrocarbyl group containing 1 to 6 carbon atoms, wherein the hydrocarbyl group is linear or branched. R₁, R₂, and R₃ may be individually selected such that not all three of R₁, R₂, and R₃ are H and the naphtha range alkane has a minimum of 5 carbon atoms. In Reaction 1, the ketone and alcohol may include R₁, R₂, and R₃ groups that correspond to the R₁, R₂, and R₃ groups present in the naphtha range alkane reactant. Any of a variety of naphtha range alkanes may be used in the oxidation of Step (1). Suitable naphtha range alkanes may have, for example, from 5 carbon atoms to 12 carbon atoms such as those previously described.

Oxidation of naphtha range alkanes may be carried out in liquid phase or in gaseous phase. In some examples, the naphtha range alkanes may be oxidized in the liquid phase via auto-oxidation. The oxidation reaction may follow a radical reaction pathway giving secondary alcohols as the primary product with the same carbon number as the corresponding naphtha range alkane the secondary alcohol was synthesized from. The produced alcohols may be reactive and therefore prone to further oxidation which may produce ketones as well as smaller oxygenate species such as carboxylic acids and aldehydes. The resultant oxygenate product stream may comprise a mixture of unreacted naphtha range alkane, and a mixture of oxygenate species including alcohols, ketones, carboxylic acids, and aldehydes.

Alternatively, oxidation selectivity to alcohol may be increased by inclusion of a boron-containing reagent such as boric acid, metaboric acid (oxoborinic acid), or borate esters in the reaction. The oxidation of naphtha range alkanes in the presence of boron compounds such as boron-containing reagent may be referred to as Bashkirov oxidation. The boron-containing reagent may react with an oxygenate species to produce a boron-oxygenate adduct species with protects the oxygenate species from further reaction. For example, in the presence of a boron reagent, the alcohol product is converted to a borate ester, thus protecting the alcohol from further oxidation. The borate ester may then be hydrolyzed which returns the alcohol and regenerates the boron reagent.

Without wishing to be bound by theory, it is believed that the alcohol formed in Reaction (1) may be trapped by the boron containing reagent by (trans)esterification to give a boron-oxygenate adduct in solution. The boron species may also promote selective decomposition of intermediate alkyl hydroperoxides in solution to the corresponding alcohol. Optionally, a radical initiator such as hydroperoxides may be used to initiate the oxidation reaction. Suitable hydroperoxides may include, but are not limited to, t-butylhydroperoxide, cumylhydroperoxide, di-t-butylperoxide, and combinations thereof.

The boron containing reagent may be a boric acid (e.g. orthoboric acid or metaboric acid), a borate ester (e.g. an orthoborate ester or a metaborate ester) or boric oxide. Borate esters may be derived from boric acids (e.g. orthoboric acid or metaboric acid) or from boric oxide in which one or more substituents are attached to the oxygen atoms. The organic groups for the borate ester, or substituents, may be selected from alkyl groups, aryl groups, benzyl groups, alkyl-carbonyls, alkyl-amides, alkyl-amines or alkyl-ethers. In some examples, the R groups are alkyl groups.

Any suitable source of oxygen may be used in the oxidation of Step (1). In some examples it may be desired that the oxygen-to-hydrocarbon vapor ratio may be maintained outside the explosive regime. For example, source of oxygen may include air (approximately 21 vol % oxygen), a mixture of nitrogen and oxygen, or pure oxygen. The mixture of nitrogen and oxygen may contain, for example, about 1 vol % to about 20 vol % oxygen (or greater).

The oxidation of Step (1) may occur in an oxidation unit which includes equipment to facilitate the oxidation reaction. The oxidation unit may include a reactor and supporting equipment to control the oxidation reaction, add reactants, remove products, and maintain and control pressure and temperature. The oxidation step may occur at any suitable oxidation conditions, including temperature, pressure, and residence time. For example, the oxidation of Step (1) may occur at a temperature of about 50° C. or greater. In some embodiments, the temperature of the oxidation may range from about 50° C. to about 200° C. or, alternatively, from about 130° C. to about 160° C. In some embodiments, the oxidation reaction may be carried out at a pressure of about 500 kPa about 10100 kPa. Alternatively, the oxidation reaction may be carried out at a pressure of about 500 kPa to about 5000 kPa, about 5000 kPa to about 7500 kPa, or about 7500 kPa to about 10100 kPa. In some embodiments, the residence time in the oxidation unit may be about 0.1 hours to about 20 hours, about 0.1 hours to about 1 hour, about 1 hour to about 5 hours, or about 5 hours to about 10 hours, or about 10 to 20 hours. The oxidation reaction may be carried out in a continuous or batch process and the residence time may be selected to give a conversion to the oxygenate product of about 10% to about 40%, or greater.

In embodiments where a boron-containing reagent is used to improve selectivity for the oxidation, a boron-oxygenate adduct hydrolysis step may be needed to regenerate the desired alcohol products. For example, the boron-oxygenate adduct produced formed during the oxidation of Step (1) may be treated with water to yield an alcohol, such as a sec-alcohol. The sec-alcohol may be fed to Step (2), where oxygenate condensation occurs. The boron-containing reagent such as boric acid or metaboric acid may be regenerated by hydrolysis and recycled to the oxidation of Step (1). The hydrolysis reaction may be performed at any suitable temperature, pressure and residence time. For example, the hydrolysis may be performed in a temperature range of about 50 ° C. to about 200° C., about 50° C. to about 150° C., or about 50° C. to about 100° C. The hydrolysis may be performed in a temperature range of about 100 kPa to about 1000 kPa or about 100 kPa to about 500 kPa. The hydrolysis may be performed with a residence time of about 0.1 to about 10 hours, about 0.1 to about 5 hours, or about 0.1 to about 1 hour. In some embodiments, the hydrolysis reaction can be catalyzed by a small amount of acid or base.

In Step (2), any suitable technique for condensation of the oxygenate species to produce condensed products may be utilized. In embodiments, the technique selected to produce condensed products may be dependent upon the composition of the oxygenate product stream produced in Step (1). For example, if the oxygenate product stream of Step (1) comprises alcohols, alcohol dehydrative dimerization or Guerbet coupling may be utilized to produce the condensed products. In alcohol dehydrative dimerization, one of the condensed products may be an olefin. In Guerbet coupling, one of the condensed products may be an alcohol, wherein the alcohol is heavier than the reactant alcohols. Alternatively, if the oxygenate product stream of Step (1) comprises alcohols and ketones, aldol condensation may be utilized to produce the condensed products. In aldol condensation, one of the condensed products may be a conjugated enone. In another embodiment where the oxygenate product steam comprises alcohols and ketones, selective hydrogenation of the alcohol/ketone mixture to alcohols followed by alcohol dehydrative dimerization or Guerbet coupling may be utilized to produce the condensed products. The selective hydrogenation may occur separately or in the same reactor utilized for the condensation reactions. In the embodiment where the selective hydrogenation occurs in the condensation reactor, a hydrogen co-feed with a H₂/oxygenates mole ratio of about 0.1 to about 5, about 0.1 to about 2, or about 0.1 to about 1 may be provided. A top portion of a catalyst bed disposed within the reactor may be loaded with a selective hydrogenation catalysts such as supported metal catalysts. Supported metal catalysts may be any catalyst which promotes hydrogenation such as those containing Co, Ni, Fe, Pt, Pd, Rh, Ru, Ir, Zn, Cu, Sn, Ga or combinations thereof which may be supported on silica, alumina, or titania, for example.

Reaction (2) illustrates an alcohol dehydrative dimerization reaction to produce a condensed product comprising an olefin.

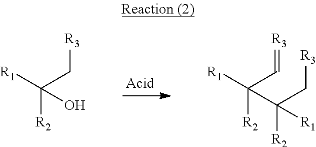

In Reaction (2) corresponding to Step (2) above, the reactant alcohol may be the alcohol produced from the oxidation reaction of Step (1). The alcohol may include R₁, R₂, and R₃ substitution groups which may correspond to the R₁, R₂, and R₃ groups from the naphtha range alkanes from Step (1). Further, the condensed product comprising an olefin may include R₁, R₂, and R₃ substitution groups which may correspond with the R₁, R₂, and R₃ groups of the reactant alcohol in Reaction (2). Although Reaction (2) is illustrated as being a reaction between two molecules of an alcohol with the same substitution groups, the alcohols may have disparate substitution groups depending on the composition of the oxygenate product stream from Step (1).

In Reaction (2), the dehydrative dimerization may carried out in the presence of an acid. Suitable acids may include, but are not limited to protic liquid acids such as sulfuric acid, hydrochloric acid, or sulfonic acid, solid acids such as acidic metal oxides including W/ZrO₂ and sulfated zirconia, amorphous aluminosilicates, acid clays, acidic resins, zeolites, silicoaluminophosphates, metal-organic frameworks (MOF), covalent organic frameworks (COF), zeolitic imidazolium frameworks (ZIF), and combinations thereof. Any suitable amount of acid catalyst may be used for catalyzing the dehydrative dimerization, including, an amount of about 0.001 mol to about 100 mol % of the total moles of reactants. Alternatively, about 0.01 mol % to about 5 mol %, about 5 mol % to about 20 mol %, about 20 mol % to about 50 mol %, or about 50 mol % to about 100 mol %.

The dehydrative dimerization Reaction (2) corresponding to Step (2) above may occur in a condensation unit which includes equipment to facilitate the dehydrative dimerization reaction. The condensation unit may include a reactor and supporting equipment to control the dehydrative dimerization reaction, add reactants, remove products, and maintain and control pressure and temperature. The dehydrative dimerization reaction may occur at any suitable conditions, including temperature, pressure, and residence time. For example, the dehydrative dimerization of Reaction (2) may occur at a temperature of about 50° C. or greater. In some embodiments, the temperature of the dehydrative dimerization reaction may range from about 50° C. to about 350° C. or greater.

Alternatively, the dehydrative dimerization may be carried out at a temperature from about 50° C. to about 150° C., or about 150° C. to about 250° C., or about 250° C. to about 350° C. In some embodiments, the dehydrative dimerization reaction may be carried out at a pressure of about 500 kPa about 10100 kPa. Alternatively, the dehydrative dimerization reaction may be carried out at a pressure of about 500 kPa to about 5000 kPa, about 5000 kPa to about 7500 kPa, or about 7500 kPa to about 10100 kPa. In some embodiments, the residence time in the condensation unit may be about 0.1 hours to about 30 hours. Alternatively, the residence may be about 0.1 hours to about 1 hours, about 1 hours to about 5 hours, about 5 hours to about 10 hours, or about 10 ours to about 30 hours. The dehydrative dimerization reaction may be carried out in a continuous or batch process.

Reaction (3) illustrates a Guerbet coupling reaction to produce a condensed product comprising a heavier alcohol.

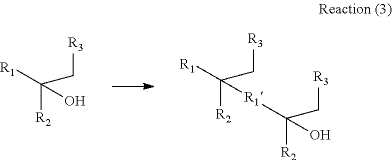

In Reaction (3) corresponding to Step (2) above, the reactant alcohol may be the alcohol produced from the oxidation reaction of Step (1). The alcohol may include R₁, R₂, and R₃ substitution groups which may correspond to the R₁, R₂, and R₃ groups from the naphtha range alkanes from Step (1). Further, the condensed product comprising a heavier alcohol may include R₁, R₂, and R₃ substitution groups which may correspond with the R₁, R₂, and R₃ groups of the reactant alcohol in Reaction (3). R₁′ group is an alkylidene group corresponding to R₁ group with one less hydrogen atom. Although Reaction (3) is illustrated as being a reaction between two molecules of an alcohol with the same substitution groups, the reactant alcohols may have disparate substitution groups depending on the composition of the oxygenate product stream from Step (1). When alcohols with disparate substitution groups are reacted, the resultant product alcohol will also contain the disparate substitution groups.

Guerbet condensation may be catalyzed by metal/base bi-functional catalysts. The reaction pathways may include 1) dehydrogenation of the alcohol to aldehyde or ketone by the metal function; 2) aldehyde or ketone aldol condensation to unsaturated ketone/aldehyde catalyzed by the base; 3) rehydrogenation of the unsaturated ketone/aldehyde to alcohol by the metal function. Some examples of suitable metal/base bi-functional catalysts may include those which comprise a metal and a base. Some suitable metals may include transition metals of Group VI and above such as, without limitation, Pt—Ga, Pt—Sn, Pt—Zn, Pt—Ag, Fe, Ru, Ni, Co, Cu, and Au, and a base that includes alkali oxides, such as, without limitation, Na₂O, K₂O, Cs₂O, and alkali earth oxides such as MgO and BaO, rare-earth oxides La₂O₃, Y₂O₃, CeO₂, and combinations thereof. Additionally, the base may be carbonates or hydroxides of group 1 or 2 metals, hydroxycarbonates of group 2-13 metals such as hydrotalcite, MgₐAl_{b}(OH)_{c}(CO₃)_{d}(a, b, c, d are the mole fractions in the formula, which can be in the range of 0.1-5, such as 0.1-3, or 0.1-2). Any suitable amount of metal/base bi-functional catalysts may be used for catalyzing the Guerbet condensation, including, an amount of about 0.001 mol % to about 5 mol % of the total moles of reactants. Alternatively, about 0.01 mol % to about 1 mol %, about 1 mol % to about 2 mol %, or about 2 mol % to about 5 mol %.

The Guerbet coupling illustrated in Reaction (3) which corresponds to Step (2) above may occur in a condensation unit which includes equipment to facilitate the Guerbet coupling reaction.

The condensation unit may include a reactor and supporting equipment to control the Guerbet coupling reaction, add reactants, remove products, and maintain and control pressure and temperature. The Guerbet coupling reaction may occur at any suitable conditions, including temperature, pressure, and residence time. For example, the Guerbet coupling of Reaction (3) may occur at a temperature of about 50° C. or greater. In some embodiments, the temperature of the dehydrative dimerization reaction may range from about 50° C. to about 350° C. or greater. Alternatively, the Guerbet coupling reaction may be carried out at a temperature from about 50° C. to about 150° C., or about 150° C. to about 250° C., or about 250° C. to about 350° C. In some embodiments, the Guerbet coupling reaction may be carried out at a pressure of about 500 kPa about 10100 kPa. Alternatively, the Guerbet coupling reaction may be carried out at a pressure of about 500 kPa to about 5000 kPa, about 5000 kPa to about 7500 kPa, or about 7500 kPa to about 10100 kPa. In some embodiments, the residence time in the condensation unit may be about 0.1 hours to about 30 hours. Alternatively, the residence may be about 0.1 hours to about 1 hour, about 1 hour to about 5 hours, about 5 hours to about 10 hours, or about 10 hours to about 30 hours. The Guerbet coupling reaction may be carried out in a continuous or batch process.

Reaction (4) illustrates an aldol condensation reaction to produce a condensed product comprising a conjugated enone.

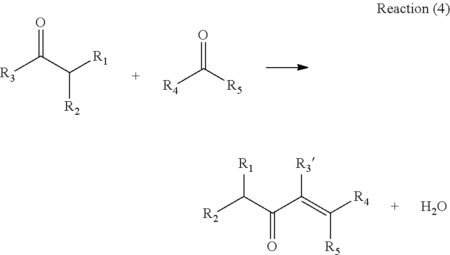

In Reaction (4) corresponding to Step (2) above, the reactants may include the ketones produced from the oxidation reaction of Step (1). The ketones may include R₁, R₂, and R₃ substitution groups which may correspond to the R₁, R₂, and R₃ groups from the naphtha range alkanes from Step (1). Additionally, Reaction (4) includes a ketone with substitution groups R₄ and R₅ which may be the same or different than the substitution groups R₁, R₂, and R₃ or may be different substation groups present in the naphtha range alkanes from Step (1). Further, the conjugated enone product may include R₁, R₂, R₃, R₄, and R₅ substitution groups which may correspond with the R₁, R₂, R3, R₄, and R₅ groups of the reactant ketones in Reaction (4). R₃′ is a substituent group corresponding to R₃ with one less carbon. Although Reaction (4) is illustrated as being a reaction between two ketone molecules with disparate substitution groups, the reactant ketones may have the same substitution groups depending on the composition of the oxygenate product stream from Step (1).

The aldol condensation Reaction (4) corresponding to Step (2) above may occur in a condensation unit which includes equipment to facilitate the aldol condensation reaction. The condensation unit may include a reactor and supporting equipment to control the aldol condensation, add reactants, remove products, and maintain and control pressure and temperature. The aldol condensation may occur at any suitable conditions, including temperature, pressure, and residence time. For example, the aldol condensation of Reaction (4) may occur at a temperature of about 50° C. or greater. In some embodiments, the temperature of the aldol condensation reaction may range from about 50° C. to about 350° C. or greater. Alternatively, the aldol condensation may be carried out at a temperature from about 50° C. to about 150° C., or about 150° C. to about 250° C., or about 250° C. to about 350° C. In some embodiments, the aldol condensation reaction may be carried out at a pressure of about 500 kPa about 10100 kPa. Alternatively, the aldol condensation reaction may be carried out at a pressure of about 500 kPa to about 5000 kPa, about 5000 kPa to about 7500 kPa, or about 7500 kPa to about 10100 kPa. In some embodiments, the residence time in the condensation unit may be about 0.1 hours to about 30 hours. Alternatively, the residence may be about 0.1 hours to about 1 hour, about 1 hours to about 5 hours, about 5 hours to about 10 hours, or about 10 hours to about 30 hours. The aldol condensation reaction may be carried out in a continuous or batch process.

The aldol condensation Reaction (4) may be catalyzed by a basic metal oxide. For example, some suitable base catalysts may include, but are not limited to, alkali oxides, hydroxides, carbonates, or bicarbonates, alkali earth oxides, hydroxides, or carbonates, rare-earth oxides, group 2 to 13 metal hydroxycarbonates such as hydrotalcites, group 2 to 13 metal carbonates, and combinations thereof. Any suitable amount of catalysts may be used for catalyzing the aldol condensation reaction, including, an amount of about 0.001 mol % to about 5 mol % of the total moles of reactants. Alternatively, about 0.01 mol % to about 1 mol %, about 1 mol % to about 2 mol %, or about 2 mol % to about 5 mol %.

In another embodiment, if the oxygenate product stream of Step (1) comprises alcohols and ketones, the oxygenate product stream may undergo selective hydrogenation of the alcohol/ketone mixture to alcohols. Thereafter, alcohol dehydrative dimerization of Reaction (2) or Guerbet coupling of Reaction (3) may be carried out on the mixture of alcohols to produce the corresponding condensed products as previously described.

In Step (3), any suitable technique for hydro-finishing of the condensed products of Step (2) to produce distillate range products may be used. The condensed products utilized in Step (3) may be a product stream from a condensation unit, described in detail above. The composition of the condensed products from Step (2) may depend upon the reaction route chosen to produce the condensed products. For example, in dehydrative dimerization the product may include an olefin, in aldol condensation the product may include a conjugated enone, and in Guerbet coupling the product may include an alcohol. By way of example, the hydro-finishing step may include hydro-finishing reactions such as reacting olefinic bonds, alcohols, and ketones with hydrogen, thereby reducing the concentration of olefins, ketones, and alcohols in the condensed products. The hydro-finished product is the distillate range product previously described. Reaction (5), Reaction (6), and Reaction (7) illustrate the addition of hydrogen to olefinic bonds, alcohols, and ketones with the resultant product being a saturated molecule of distillate range product.

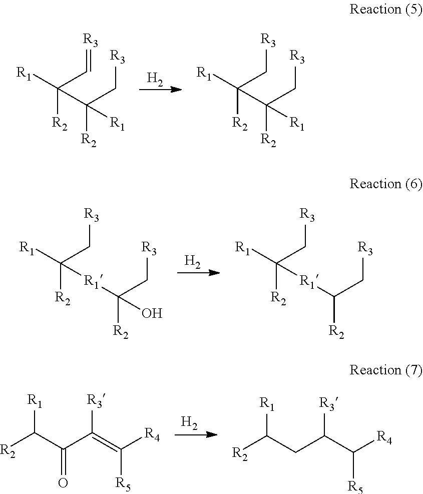

The hydro-finishing illustrated in Reaction (5), Reaction (6), and Reaction (7) may correspond to Step (3) above. The hydro-finishing reactions may occur in a hydro-finishing unit which includes equipment to facilitate the hydro-finishing reactions. The hydro-finishing reactions may include any reactions where hydrogen is added to a molecule. The hydro-finishing unit may include a reactor and supporting equipment to control the hydro-finishing reaction, add reactants, remove products, and maintain and control pressure and temperature. The hydro-finishing reactions may occur at any suitable conditions, including temperature, pressure, and residence time. For example, the hydro-finishing condensation of Reaction (5), Reaction (6), and Reaction (7) may occur at a temperature of about 50° C. or greater. In some embodiments, the temperature of the hydro-finishing reaction may range from about 50° C. to about 350° C. or greater. Alternatively, the hydro-finishing reactions may be carried out at a temperature from about 50° C. to about 150° C., or about 150° C. to about 250° C., or about 250° C. to about 350° C. In some embodiments, the hydro-finishing reaction may be carried out at a pressure of about 500 kPa about 10100 kPa. Alternatively, the hydro-finishing reaction may be carried out at a pressure of about 500 kPa to about 5000 kPa, about 5000 kPa to about 7500 kPa, or about 7500 kPa to about 10100 kPa. In some embodiments, the residence time in the hydro-finishing unit may be about 0.1 hours to about 30 hours. Alternatively, the residence may be about 0.1 hours to about 1 hour, about 1 hour to about 5 hours, about 5 hours to about 10 hours, or about 10 hours to about 30 hours. The hydrogen to feed ratio is in the range of 1-100, such as 1-50, or 1-25. The hydro-finishing reaction may be carried out in a continuous or batch process.

The hydro-finishing reactions such as those illustrated in Reaction (5), Reaction (6), and

Reaction (7), for example, may be catalyzed by a hydrogenation catalyst. Some suitable hydrogenation catalysts may include, without limitation, late transition metals, such as Group VI and above, supported on alumina, silica, zirconia, titania, or carbon, for example. Example of the metal may include, without limitation, Cr, Mo, Mn, Re, Fe, Co, Ni, Pt, Pd, Ru, Rh, Ir, Au, Ag, Cu, Zn, Ga, and Sn. Any suitable amount of catalyst may be used for catalyzing the hydro-finishing reactions, including, an amount of about 0.001 mol % to about 5 mol % of the total moles of reactants. Alternatively, about 0.01 mol % to about 1 mol %, about 1 mol % to about 2 mol %, or about 2 mol % to about 5 mol %.

An embodiment will now be described in detail for producing distillate range products from n-heptane via oxygenate intermediates. In the present embodiment, the naphtha range alkane is n-heptane which may be considered to have to have R₁ and R₂ hydrogen substitution and R₃ pentyl substitution. A first step in the present embodiment may include oxidation of n-heptane as illustrated in Reaction (8), corresponding to Step (1) described above.

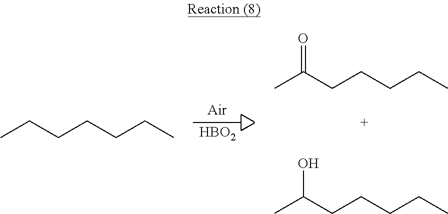

In Reaction (8), the oxidation of n-heptane may proceed according the any of the methods described above. Reaction (8) illustrates Bashkirov oxidation by inclusion of metaboric acid to promote selectivity to 2-heptanol over 2-heptanone. The oxidation reaction may be carried out in and oxidation unit as described above and an oxygenate product stream comprising unreacted n-heptane, as well as 2-heptanol and 2-heptanone may exit the oxidation unit.

A second step in the present embodiment may include the reaction of species in the oxygenate product stream to produce condensed products. Reaction (9), corresponding to Step (2) above, illustrates the 2-heptanone produced from Reaction (8) being catalytically reacted using Guerbet coupling and aldol condensation to form condensed species. Reaction (10), also corresponding to Step (2) above, illustrates the 2-heptanol from Reaction (8) undergoing dehydrative dimerization to produce a condensed product. Reaction (9) and Reaction (10) may proceed according to any of the methods described above and may be carried out in a condensation unit as described above.

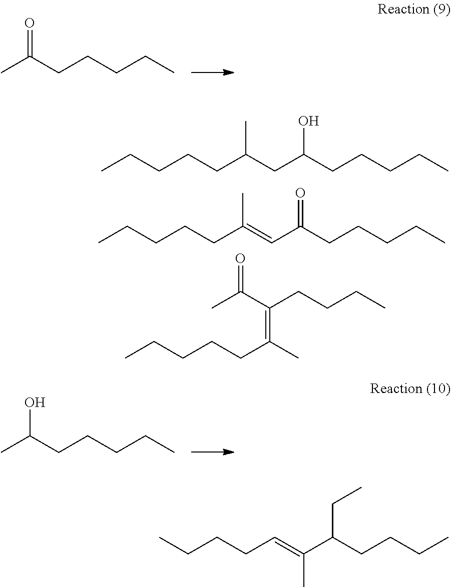

A third step in the present embodiment may include reaction of the condensed product from Reaction (9), Reaction (10), or both with hydrogen to hydro-finish the condensed product to form the distillate range product. Reaction (11), corresponding to Step (3) above, illustrates the addition of hydrogen to the products of Reaction (9) to form a distillate range product. Reaction (12), corresponding to Step (3) above, illustrates the addition of hydrogen to the product of Reaction (10) to form a distillate range product. Reaction (11) and Reaction (12) may proceed according to any of the methods described above and may be carried out in a hydro-finishing unit as described above.

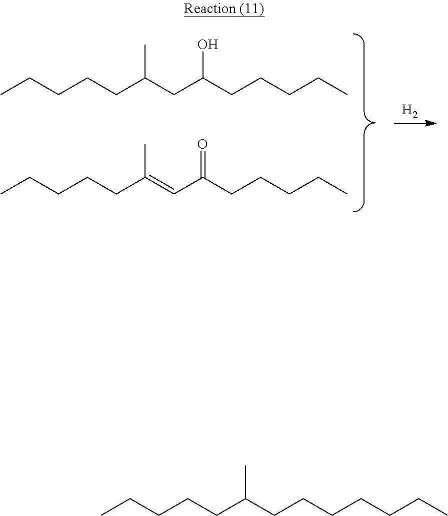

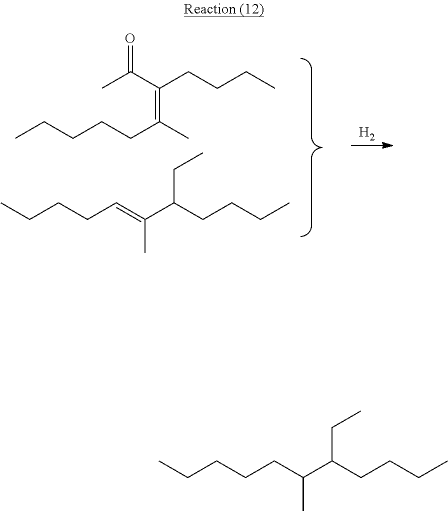

The FIGURE is a flow chart illustrating a process 100 to produce distillate range products from naphtha range alkanes. Process 100 may comprise three primary processes, oxidation, condensation, and hydro-finishing. As illustrated in the FIGURE, process 100 may begin by introducing naphtha range alkane feed 101 to oxidation unit 102. Naphtha range alkane feed 101 may comprise any of the naphtha range alkanes previously described, including those with carbon numbers ranging from C₅ to C₁₂ and may be from any source, including processing units not explicitly shown in the FIGURE. An oxygen feed 109 and boron reagent feed 110 may also be introduced into oxidation unit 102 as previously described. Oxidation unit 102 may comprise a reactor and catalyst where the reactions of Step 1 may be performed. Oxidation unit 102 may oxidize at least a portion of naphtha range alkane feed 101 to produce oxygenate products. The oxygenate products may include any of the oxygenate products disclosed herein including alcohols and ketones, for example. Oxidized effluent stream 103 may comprise the oxygenate products including the alcohols and ketones as well as unreacted naphtha range alkanes from naphtha range alkane feed 101 and any air or oxygen. In examples where Bashkirov reagents such as boron compounds are utilized, an adduct stream 112 may exit oxidation unit 102 and be sent to a boron-oxygenate adduct hydrolysis unit 111. A water stream 114 may be introduced into boron-oxygenate adduct hydrolysis unit 111 which may react with boron adducts present to generate oxidized effluent stream 115 and recycle boron reagent feed 113. Oxidized effluent stream 115 may exit boron-oxygenate adduct hydrolysis unit 111 and be introduced into condensation unit 104

Oxidized effluent stream 103 may exit oxidation unit 102 and be introduced into condensation unit 104. Although not illustrated in the FIGURE, oxidized effluent stream 103 may be introduced into a separation unit (not illustrated) whereby a portion of the oxygenate product stream is removed before introduction into condensation unit 104. For example, at least a portion of oxygen, nitrogen, water, and/or Bashkirov reagents may be removed from oxidized effluent stream 103 prior to introduction into condensation unit 104. Further, the oxygenate product stream 103 may be introduced into a selective hydrogenation unit (not illustrated) whereby at least a portion of the ketones present in oxygenate product steam are converted to alcohols prior to introduction into condensation unit 104.

Condensation unit 104 may comprise a reactor and catalyst whereby the reactions of Step (2), described above, may be performed. Condensation unit 104 may react the oxygenate products from oxygenate product stream 103 to form condensed species as described above. The reactions, and therefore products present in condensation unit 104 may depend on the composition of the feed to condensation unit 104. Condensed product stream 105 comprising the condensed species produced in condensation unit 104 may exit condensation unit 104 and be introduced to hydro-finishing unit 106. Hydro-finishing unit 106 may comprise a reactor and catalyst whereby the reactions of Step (3), described above, may be performed. Hydro-finishing unit 106 may react hydrogen from hydrogen stream 108 with the condensed products from condensed product stream 105 to produce a distillate range product stream 107.

Accordingly, the preceding description describes production of distillate range products from naphtha range alkanes with oxygenate intermediates. The systems and methods disclosed herein may include any of the various features disclosed herein, including one or more of the following embodiments.

Embodiment 1. A method comprising: reacting at least a naphtha range alkane with oxygen and to produce oxygenate products; reacting at least a portion of the oxygenate products to produce condensed products; and reacting at least a portion of the condensed products with at least hydrogen to produce a distillate range product.

Embodiment 2. The method of embodiment 1 wherein the step of reacting at least the naphtha range alkane with oxygen comprises reacting the naphtha range alkane in the presence of a boron containing species to produce a boron-oxygenate adduct.

Embodiment 3. The method of embodiment 2 further comprising hydrolyzing at least a portion of the boron-oxygenate adduct.

Embodiment 4. The method of any of embodiments 1-3 wherein the naphtha range alkane comprises naphtha with carbon numbers ranging from C5 to C12.

Embodiment 5. The method of any of embodiments 1-4 further comprising at least partially hydrogenating the oxygenate products before the step of reacting at least a portion of the oxygenate products.

Embodiment 6. The method of any of embodiments 1-5 wherein the step of reacting at least a portion of the oxygenate products comprises at least one of dehydrative dimerization, aldol condensation, Guerbet coupling, or a combination thereof.

Embodiment 7. The method of any of embodiments 1-6 wherein the step of reacting at least a portion of the condensed products with hydrogen comprises reacting at least one of olefinic bonds, ketones, or alcohols present in the condensed products with hydrogen.

Embodiment 8. The method of any of embodiments 1-7 wherein the distillate range product comprises hydrocarbons with carbon numbers ranging from C10 to C36.

Embodiment 9. A method comprising: introducing a naphtha range alkane feed into an oxidation unit wherein the naphtha range alkane feed comprises naphtha with a boiling point of about 30° C. to about 200° C. at atmospheric pressure; reacting at least the naphtha with oxygen to produce an oxygenate product comprising at least one of an alcohol, a ketone, or combinations thereof; introducing the oxygenate product into a condensation unit; reacting at least a portion of the oxygenate product to produce a condensed product comprising at least one of an olefin, an alcohol, a conjugated enone, or a combination thereof; introducing the condensed products into a hydro-finishing unit; and reacting at least a portion of at least the condensed products with hydrogen to produce a distillate range product.

Embodiment 10. The method of embodiment 9 wherein the naphtha comprises hydrocarbons with carbon numbers ranging from C5 to C12.

Embodiment 11. The method of any of embodiments 9-10 wherein the step of reacting at least a portion of the naphtha with oxygen comprises reacting the naphtha in the presence of a boron-containing reagent to produce a boron-oxygenate adduct.

Embodiment 12. The method of embodiment 11 further comprising hydrolyzing at least a portion of the boron-oxygenate adduct prior to the step of introducing the oxygenate product into a condensation unit.

Embodiment 13. The method of any of embodiments 9-12 wherein the step of reacting at least a portion of the oxygenate product to produce a condensed product comprises at least one of dehydrative dimerization, aldol condensation, Guerbet coupling, or a combination thereof.

Embodiment 14. The method of embodiment 13 wherein the step of reacting comprises dehydrative dimerization or Guerbet coupling, and wherein the method further comprises at least partially hydrogenating the oxygenate product before the step of reacting at least a portion of the oxygenate products.

Embodiment 15. The method of any of embodiments 13-14 wherein the step of reacting at least a portion of the condensed products with hydrogen comprises reacting at least one of olefinic bonds, ketones, or alcohols present in the condensed products with hydrogen.

Embodiment 16. The method of any of embodiments 13-15 wherein the distillate range product comprises hydrocarbons with carbon numbers ranging from C10 to C36.

Embodiment 17. A system comprising: an oxidation unit, wherein a naphtha range alkane feed is coupled to one or more inputs of the oxidation unit, and wherein the oxidation unit is configured to oxidize at least a portion of the naphtha range alkane feed to produce an oxidized effluent stream comprising oxygenates; a condensation unit, wherein the oxidized effluent stream from the oxidation unit is coupled to one or more inputs of the condensation unit, and wherein the condensation unit is configured to react at least a portion of the oxygenates from the oxidized effluent stream to produce a condensed product stream comprising condensed products; and a hydro-finishing unit, wherein the condensed product stream effluent from the condensation unit and a hydrogen stream are coupled to one or more inputs of the hydro-finishing unit, and wherein the hydro-finishing unit is configured to react at least a portion of the condensed products with hydrogen to produce a distillate range product stream.

Embodiment 18. The system of embodiment 17 wherein a boron-containing reagent feed is coupled to one or more inputs of the oxidation unit.

Embodiment 19. The system of any of embodiments 17-18 wherein the condensation unit comprises a metal/base bi-functional catalyst configured to catalyze a Guerbet coupling reaction or a base catalyst configured to catalyze an aldol condensation reaction.

Embodiment 20. The system of any of embodiments 17-19 wherein the condensation unit comprises an acid catalyst configured to dehydrate C5 to C12 alcohols to C5 to C12 olefins and dimerize the C5 to C12 olefins to C10 to C24 olefins.

### EXAMPLES

To facilitate a better understanding of the present disclosure, the following examples of certain aspects of some embodiments are given. In no way should the following examples be read to limit, or define, the entire scope of the disclosure.

### Example 1

In this example, an MCM-49 based catalyst suitable for catalyzing the reactions disclosed herein is prepared. 80 parts of MCM-49 zeolite crystals and 20 parts pseudoboehmite alumina on a calcined dry weight basis were weighed and added to a mixer. The MCM-49 and pseudoboehmite alumina were mixed for a period of about 10 to about 30 minutes. A sufficient amount of a solution of water and 0.05% polyvinyl alcohol was added to the mixture to form an extrudable paste. The extrudable paste was formed into a 1/20 inch quadralobed extrudate using an extruder. After extrusion, the extrudate was dried at a temperature ranging from about 250° F. (121° C.) to about 325° F. (168° C.). After drying the extrudate was heated at 1000° F. (538° C.) under flowing nitrogen. The extrudate was then cooled to ambient temperature and humidified with saturated air or steam. After humidification, the extrudate was ion exchanged with 0.5 to 1 N (normal) ammonium nitrate solution. The ammonium nitrate treated extrudate was then washed with de-ionized water to remove residual nitrate and dried. After drying, the extrudate was calcined in a nitrogen/air mixture at a temperature to 1000° F. (538° C.).

### Example 2

In this example, a zeolite beta based catalyst suitable for catalyzing the reactions disclosed herein is prepared. 65 parts of zeolite beta crystals and 35 parts pseudoboehmite alumina on a calcined dry weight basis were weighed and added to a mixer. The zeolite beta and pseudoboehmite alumina were mixed for a period of about 10 to about 30 minutes. A sufficient amount of a solution of water was added to the mixture to form an extrudable paste. The extrudable paste was formed into a 1/16 inch quadralobed extrudate using an extruder. After extrusion, the extrudate was dried at a temperature ranging from about 250° F. (121° C.) to about 325° F. (168° C.). After drying the extrudate was heated at 1000° F. (538° C.) under flowing nitrogen. The extrudate was then cooled to ambient temperature and humidified with saturated air or steam. After humidification, the extrudate was ion exchanged with 0.5 to 1 N (normal) ammonium nitrate solution. The ammonium nitrate treated extrudate was then washed with de-iniozed water to remove residual nitrate and dried. After drying, the extrudate was calcined in a nitrogen/air mixture at a temperature to 1000° F. (538° C.). After calcination, the extrudate was steamed at 700° F. (371° C.) for 3 hours.

### Example 3

In this example, the MCM-49 based catalyst and zeolite beta based catalyst were utilized in a heptanol dehydration/dehydrative coupling reaction. A reactor comprising a stainless steel tube with dimensions of ⅜ in [i.d.] (9.525 mm)×20.5 in (520.7 mm)×0.035 in (0.889 mm) wall thickness was prepared. A piece of stainless steel tubing 8¾ in. (222.25 mm) long×⅜ in. (9.525 mm) o.d. and a piece of ¼ in (6.35 mm) tubing of similar length was used in the bottom of the reactor as a spacer (one inside of the other) to position and support the catalyst in the isothermal zone of the furnace. A ⅛ in (3.175) stainless steel thermo-well was placed in the catalyst bed, long enough to monitor temperature throughout the catalyst bed using a movable thermocouple. About 5cc of catalyst was sized to about 14-25 mesh (710 micrometer) and diluted with quartz.

The catalyst was loaded into the reactor until the catalyst bed measured about 10 cm in height. A ¼ in (6.35 mm) plug of glass wool was placed at the top of the spacer to keep the catalyst in place and a ¼ in (6.35 mm) plug of glass wool was placed at the top of the catalyst bed to separate quartz chips from the catalyst. The remaining void space at the top of the reactor was filled with quartz chips. The reactor was installed in the furnace with the catalyst bed in the middle of the furnace at the pre-marked isothermal zone. The reactor was then pressure and leak tested, to about 800 psig (5516 kPa).

A blend of 50:50 2- and 3- heptanol was pumped into the reactor. A back pressure controller was used to control the reactor pressure which was set at 350-750 psig (2413-5171 kPa). On-line GC (gas chromatography) analyses were taken to verify feed and the product composition. The blend pumped through the catalyst bed held at the reaction temperature of 150-250° C. The products exiting the reactor flowed through heated lines routed to the online GC sample location, then to chilled collection pots. The non-condensable gas products exiting the chilled collection pots overhead vents were routed through a gas pump for analysis. Samples from the gas and liquid were taken for analysis. Data from the reactor effluent online GC, vent gas online GC, and collection pots samples were combined to perform material balances at 24 hour intervals. The results are shown in the Table 1 below.

TABLE 1

| Catalyst | MCM-49 | Beta | Beta | Beta |
|---|---|---|---|---|
| T ° C. | 200 | 170 | 190 | 200 |
| Feed Rate cc/h | 1.5 | 2.5 | 1.5 | 0.75 |
| Sum of products lighter | 1.13 | 0.36 | 1.21 | 1.29 |
| than C7, % | | | | |
| Heptenes % | 95.31 | 91.8 | 88.88 | 88.87 |
| Heptanols % | 3.23 | 5.3 | 4.72 | 3.44 |
| C14+ % | 0.32 | 2.54 | 5.19 | 6.41 |

While the invention has been described with respect to a number of embodiments and examples, those skilled in the art, having benefit of this disclosure, will appreciate that other embodiments can be devised which do not depart from the scope and spirit of the invention as disclosed herein. Although individual embodiments are discussed, the invention covers all combinations of all those embodiments.

While compositions, methods, and processes are described herein in terms of “comprising,” “containing,” “having,” or “including” various components or steps, the compositions and methods can also “consist essentially of” or “consist of” the various components and steps. The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of the invention, additionally, they do not exclude impurities and variances normally associated with the elements and materials used.

All numerical values within the detailed description and the claims herein modified by “about” or “approximately” with respect the indicated value are intended to take into account experimental error and variations that would be expected by a person having ordinary skill in the art.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited.

1. A method comprising:
reacting at least a naphtha range alkane with oxygen and to produce oxygenate products;
reacting at least a portion of the oxygenate products to produce condensed products; and
reacting at least a portion of the condensed products with at least hydrogen to produce a distillate range product.

2. The method of claim 1 wherein the step of reacting at least the naphtha range alkane with oxygen comprises reacting the naphtha range alkane in the presence of a boron containing species to produce a boron-oxygenate adduct.

3. The method of claim 2 further comprising hydrolyzing at least a portion of the boron-oxygenate adduct.

4. The method of claim 1 wherein the naphtha range alkane comprises naphtha with carbon numbers ranging from C₅ to C₁₂.

5. The method of claim 1 further comprising at least partially hydrogenating the oxygenate products before the step of reacting at least a portion of the oxygenate products.

6. The method of claim 1 wherein the step of reacting at least a portion of the oxygenate products comprises at least one of dehydrative dimerization, aldol condensation, Guerbet coupling, or a combination thereof.

7. The method of claim 1 wherein the step of reacting at least a portion of the condensed products with hydrogen comprises reacting at least one of olefinic bonds, ketones, or alcohols present in the condensed products with hydrogen.

8. The method of claim 1 wherein the distillate range product comprises hydrocarbons with carbon numbers ranging from C₁₀ to C₃₆.

9. A method comprising:
introducing a naphtha range alkane feed into an oxidation unit wherein the naphtha range alkane feed comprises naphtha with a boiling point of about 30° C. to about 200° C. at atmospheric pressure;
reacting at least the naphtha with oxygen to produce an oxygenate product comprising at least one of an alcohol, a ketone, or combinations thereof;
introducing the oxygenate product into a condensation unit;
reacting at least a portion of the oxygenate product to produce a condensed product comprising at least one of an olefin, an alcohol, a conjugated enone, or a combination thereof;
introducing the condensed products into a hydro-finishing unit; and
reacting at least a portion of at least the condensed products with hydrogen to produce a distillate range product.

10. The method of claim 9 wherein the naphtha comprises hydrocarbons with carbon numbers ranging from C₅ to C₁₂.

11. The method of claim 9 wherein the step of reacting at least a portion of the naphtha with oxygen comprises reacting the naphtha in the presence of a boron-containing reagent to produce a boron-oxygenate adduct.

12. The method of claim 11 further comprising hydrolyzing at least a portion of the boron-oxygenate adduct prior to the step of introducing the oxygenate product into a condensation unit.

13. The method of claim 9 wherein the step of reacting at least a portion of the oxygenate product to produce a condensed product comprises at least one of dehydrative dimerization, aldol condensation, Guerbet coupling, or a combination thereof.

14. The method of claim 13 wherein the step of reacting comprises dehydrative dimerization or Guerbet coupling, and wherein the method further comprises at least partially hydrogenating the oxygenate product before the step of reacting at least a portion of the oxygenate products.

15. The method of claim 13 wherein the step of reacting at least a portion of the condensed products with hydrogen comprises reacting at least one of olefinic bonds, ketones, or alcohols present in the condensed products with hydrogen.

16. The method of claim 13 wherein the distillate range product comprises hydrocarbons with carbon numbers ranging from C₁₀ to C₃₆.

17. A system comprising:
an oxidation unit, wherein a naphtha range alkane feed is coupled to one or more inputs of the oxidation unit, and wherein the oxidation unit is configured to oxidize at least a portion of the naphtha range alkane feed to produce an oxidized effluent stream comprising oxygenates;
a condensation unit, wherein the oxidized effluent stream from the oxidation unit is coupled to one or more inputs of the condensation unit, and wherein the condensation unit is configured to react at least a portion of the oxygenates from the oxidized effluent stream to produce a condensed product stream comprising condensed products; and
a hydro-finishing unit, wherein the condensed product stream effluent from the condensation unit and a hydrogen stream are coupled to one or more inputs of the hydro-finishing unit, and wherein the hydro-finishing unit is configured to react at least a portion of the condensed products with hydrogen to produce a distillate range product stream.

18. The system of claim 17 wherein a boron-containing reagent feed is coupled to one or more inputs of the oxidation unit.

19. The system of claim 17 wherein the condensation unit comprises a metal/base bi-functional catalyst configured to catalyze a Guerbet coupling reaction or a base catalyst configured to catalyze an aldol condensation reaction.

20. The system of claim 17 wherein the condensation unit comprises an acid catalyst configured to dehydrate C₅ to C₁₂ alcohols to C₅ to C₁₂ olefins and dimerize the C₅ to C₁₂ olefins to C₁₀ to C₂₄ olefins.
